(19) United States
(12) Patent Application Publication
(10) Pub. No.: US 2013/0205959 A1
(43) Pub. Date: Aug. 15, 2013

(54) Composition Dispensing Device Comprising A Moisturizing Composition

(71) Applicants: Neil John Jones, Staines (GB); Alison Fiona Stephens, Maidenhead (GB); James Robert Schwartz, West Chester, OH (US)

(72) Inventors: Neil John Jones, Staines (GB); Alison Fiona Stephens, Maidenhead (GB); James Robert Schwartz, West Chester, OH (US)

(21) Appl. No.: 13/586,365

(22) Filed: Aug. 15, 2012

Related U.S. Application Data
(60) Provisional application No. 61/524,078, filed on Aug. 16, 2011.

(51) Int. Cl. B26B 21/44; A61K 8/04; A61Q 9/02; B26B 21/40

(52) U.S. Cl. CPC B26B 21/446; B26B 21/4081; A61K 8/042; A61Q 9/02
USPC 83/22; 30/41; 424/73

(57) ABSTRACT

A composition dispensing device containing a moisturizing personal care composition suitable for use on wet skin.

### CROSS REFERENCE TO RELATED APPLICATION

This application claims priority to U.S. Provisional Application No. 61/524,078 filed Aug. 16, 2011, the subject of which is hereby incorporated by reference in its entirety.

### BACKGROUND OF THE INVENTION

Several different composition dispensing razors are known. See, e.g., U.S. Pat. Nos. 7,007,389; 6,308,413; 4,753,006; 4,635,361; 6,986,207; 5,855,066; and 4,129,942. These and other dispensing razors have been described as being capable of dispensing various types of shaving related preparations, including clear or translucent shaving gels or lotions.

For example, composition dispensing shavers having a plurality of shaver heads with rotary blade cutters capable of dispensing a lubricant for allegedly decreasing friction between the shaving heads and cutters with skin has been disclosed in U.S. Patent Publ. No. 2008/0216322 and RE038934. Additionally, Phillips Norelco recently marketed a composition dispensing razor under the name of Cool Skin Shaver®. This shaver has a large main head containing multiple rotary blades and dispenses a Nivea For Men® moisturizing shave lotion out of the center of the rotary blades, allowing the composition to coat the shaving head and cutters to allegedly decrease friction with skin. Kits comprising razors and moisturizing compositions have also been disclosed. See e.g. US Publication No. 2007/0137042 and 2008/0317697.

A runny or less viscous formulation may be desirable in certain instances, such as where the formulator wants the composition to dispense in a discrete area but quickly spread to contact and/or coat a large surface, such as the shaving head and cutters. It can also be desirable, however, for the product to be sufficiently thick so it will not run off or otherwise be pushed away from the portion of skin desired for treatment. Many different types of thickeners and viscosity modifying agents can impact the viscosity and rheology of the composition. Many of these ingredients, however, also impact other characteristics of the composition when added, such as making the composition stringy or tacky, or making the composition cloudy or opaque. Examples of numerous clear and/or non-foaming skin care compositions of varying thickness and viscosity are known. See e.g. WO 93/18740; GB 2236760; U.S. Pat. Nos. 2,833,693; 3,072,536; 4,585,650; 4,917,844; and 6,627,185.

One class ingredients which is believed to provide desirable thickening benefits as well as cleansing and lathering benefits includes surfactants. Many of these surfactants, however, are capable of causing undesirable skin irritation during and following use in certain instances. This can be particularly relevant where users do not wash off the composition from skin following the hair removal process. Further, many foaming compositions can also cause visibility of the surface to be obscured as a result of the foam.

The number of combinations of devices and compositions is numerous. In addition, if one were to further consider the many different types of personal care compositions which can be used, the number of executions can be near limitless. Despite the near limitless number of potential combinations of features, there remains a need for a composition dispensing device capable of dispensing a composition which is sufficiently thick and viscous yet is not undesirably cloudy or opaque.

### SUMMARY OF THE INVENTION

One aspect of the present invention provides for a composition dispensing device comprising: a first chamber and a moisturizing personal care composition contained within said first chamber, said moisturizing personal care composition comprising: an oil-containing composition comprising: a) an aqueous phase; b) from about 1 to about 80 weight percent of a structured oil phase; c) a gel-network phase comprising a non-ionic hydrophilic surfactant and a hydrophobic structuring agent; and d) an aqueous phase stability agent wherein said structured oil phase has a shear index of less than 0.8.

In one embodiment, the composition dispensing device comprises a handle connected to a hair removal head, the handle further comprising a cavity for housing said moisturizing personal care composition disposed within the handle, and an actuator adapted to displace moisturizing personal care composition from the cavity to a fluid dispensing member, and wherein said fluid dispensing member comprises an elongated elastomeric contact region forming at least one dispensing orifice which is generally perpendicular to a transverse centerline of the handle.

Another aspect provides for a method of removing hair from skin comprising the steps of: providing a composition dispensing device containing the moisturizing personal care composition of the present invention; actuating said composition dispensing device to dispense said composition; contacting said composition onto a portion of skin to be treated to form a prepared surface; and contacting said prepared surface with the razor blade of the composition dispensing device to form a treated surface.

### BRIEF DESCRIPTION OF THE DRAWINGS

FIG. 1 is a side view of a composition dispensing device suitable for use with the moisturizing personal care composition in accordance with at least one embodiment of the present invention. FIGS. 2 and 3 are side views of other embodiments suitable for use as the composition dispensing device. FIG. 4 is a side view of the composition dispensing device shown in FIG. 3, but where the device is in use on a segment of skin; FIGS. 5, 6 and 7 are various bottom planar views composition dispensing devices in accordance with one or more embodiments of the present invention. FIG. 8 is an exploded side view of portion of a fluid dispensing member. FIG. 9 is a planar view of the contact region of the fluid dispensing member of FIG. 8.

### DETAILED DESCRIPTION OF THE INVENTION

It has importantly been found that when selecting a composition to be used in a composition dispensing device, it can be particularly desirable to select a composition which is sufficiently thick and viscous that it will not run off the skin after being dispensed. Additionally, moisturizing compositions can be desirable for use in a composition dispensing razor to allow for multiple benefits, including but not limited to hydration of the hairs prior to shaving, moisturization of skin during the hair removal process, lubrication of skin to reduce friction during the shave, and so forth. Those of skill in the art will understand that moisturization can include hydration of the skin or hair or lubrication of the hair or skin to increase glide and reduce friction between the composition dispensing device and skin.

1. Moisturizing Personal Care Composition:

The moisturizing personal care composition of the present invention are selected from the group consisting of skin compatibles oils, high internal phase emulsions, oil in water emulsions, gel networks, non-aqueous systems, and polyerically stabilized dispersions and mixtures thereof. Examples of moisturizing personal care composition are described in detail in U.S. Publication No. 2004/0223992, U.S Publication No. 2003/0190296, U.S Publication No. 2003/0054019, U.S Publication No. 2003/0049282, U.S. Pat. No. 6,699,488 and U.S. Pat. No. 6,645,511.

The moisturizing personal care composition can comprise a skin compatible oil which can be present at levels by weight of the moisturizing personal care composition of: less than about 80%, less than about 70%; less than about 60% and less than about 50%. In one aspect, the moisturizing personal care composition comprises a skin compatible oil by weight of the total moisturizing personal care composition at levels of: at least about 1%, at least about 5%, at least about 7%, and at least about 10%.

The moisturizing personal care composition can comprise an oil in water emulsion that comprises a skin compatible oil and an aqueous phase. In the preferred embodiment the oil in water emulsion comprises a continuous aqueous phase, a discontinuous structured oil phase, and an aqueous phase stability agent. In one aspect, these oil and water emulsions are effectively ‘non-lathering’. In one aspect, the moisturizing personal care composition can comprise less than about 2%, less than about 1.5%, less than about 1.0%, and less than about 0.5%, by weight of the moisturizing personal care composition, of an anionic surfactant.

The structured oil phase of an oil in water emulsion comprises two essential components: a skin compatible oil and a structurant. The structurant must satisfy two requirements. First, the structurant must be capable of forming a stable network in the skin compatible oil phase at a temperature below 35° C., so that the structured oil is active during use, but is not perceived as gritty. By stable network, we mean the network survives at least one month of storage at 25° C. and 35° C. The second requirement is that the structurant provides the structured oil phase with the correct rhelogical properties. The structured oil phase should have a viscosity in the range of 100 to about 200,000 poise measured at 1 Sec-1, in one aspect 200 to about 100,000 poise, and most preferably 200 to about 50,000 poise as determined using the lipid rheology method described in U.S. Publication No. 2004/0223992. The amount of structurant required to produce this viscosity will vary depending on the type of oil and the type of structurant. In general, the amount of structurant will be less than about 75%, less than about 50%, and less than about 35%, by weight of the structured oil phase. Structurants meeting the above requirements can form a 3-dimensional network to build up the viscosity of the skin compatible oil. It has been found that such structured oil phases with the 3-dimensional network, are extremely desirable for use as wet-skin treatment compositions used in bathing. It is believed that structured oil phases have an in-use property of depositing effectively on skin and have the after use properties of being retained effectively on wet skin even after rinsing and drying to provide long-lasting after wash skin benefits without causing a too oily/greasy wet and dry feel. It is believed that these in-use and after-use properties are due to the shear thinning rheological properties and the weak structure of the network. Due to its high low-shear viscosity, structured oil can stick and retain well on the skin during application. After being deposited on the skin, the network yields easily during rubbing due to the weak structuring of the crystal network and its lower high-shear viscosity.

The degree of shear-thinning (which is described in the Lipid Rheology Method described in U.S. Publication No. 2004/0223992) exhibited by the structured oil phase is given by the value of n from the Power Law Model. For the present invention, it is preferred that the structured oil phase have a shear index less than 0.8, more preferably less than 0.6, even more preferably less than 0.5 and most preferably less than 0.4.

The structurants can be an organic structurant that is either crystalline solids or amorphous gels with molecular weight less than 5,000 Daltons, preferably less than 3,000 Daltons. Preferred organic structurants have a melting point greater than 35° C., preferably greater than 40° C. Especially preferred structurants are those that can form a solution with the selected skin compatible oil at a temperature higher than their melting point to form a free flowing clear solution. Upon cooling to the ambient temperature, the organic structurant precipitate from the oil phase to form a 3-dimensional structure providing the physical properties set forth above. Examples of organic thickeners suitable for the invention are solid fatty acid esters, natural or modified fats, fatty acid, fatty amine, fatty alcohol, natural and synthetic waxes, and petrolatum. Examples of these structurants are further described in U.S. Publication No. 2004/0223992, U.S Publication No. 2003/0190296, U.S Publication No. 2003/0054019, U.S Publication No. 2003/0049282, U.S. Pat. No. 6,699,488 and U.S. Pat. No. 6,645,511.

Moisturizing personal care composition can comprise one or more aqueous phase stability agent. The aqueous phase stability agent is a polymeric stabilizer. In one aspect, the moisturizing personal care composition can comprise levels of polymeric stabilizer, by weight of the moisturizing personal care composition, of less than about 10%, less than 8%, and less than 7%. In one aspect, the moisturizing personal care composition can comprise levels of polymeric stabilizer, by weight of the moisturizing personal care composition, of: at least 0.01%, at least 0.05%, and at least 0.1%. It is believed that polymer stabilizer builds viscosity of the moisturizing personal care composition. This can be measured using the Polymeric Stabilizer Viscosity Test as described in U.S. Publication No. 2004/0223992. Preferably, the stability agent produces a viscosity in this test of at least 1000 cps, more preferably at least 1500 cps, and still more preferably at least 2000 cps. Non-limiting examples of polymeric stabilizers useful herein include carboxylic acid polymers, polyacrylamide polymers, crosslinked polyacrylate polymers, polysaccharides, gums, modified starches. Examples of these polymeric stabilizers are further described in U.S. Publication No. 2004/0223992 and U.S. Pat. No. 6,699,488

The moisturizing personal care composition can comprise a gel comprising a hydrophobic structuring agent and a non-ionic, hydrophilic surfactant. Without being bound by theory, it is believed that the gel-network allows for good application of the product in the wet environment of bathing, allowing the product to be spread easily and deposited evenly on the skin of the user. Higher levels of gel-network interfere with deposition, effectively releasing fewer lipids from the composition and resulting more lipid rinse-off lowering deposition efficiency. Without a gel-network in the product, the product would consist essentially of water, an aqueous phase stability agent and lipid, thus the application characteristics of the product would be undesirable in that the product would be difficult to spread and deposit evenly. For this reason, when the gel-network is present the level of gel-network is kept relatively low. In a preferred embodiment, the gel-network will not form a homogeneous aqueous phase as described in the gel-network stability test described in U.S. Publication No. 2004/0223992.

Hydrophobic structuring agent is one component of the gel-network. The hydrophobic, structuring agent can be selected from the group consisting of saturated C₁₆ to C₃₀ fatty alcohols, saturated C₁₆ to C₃₀ fatty alcohols containing from about 1 to about 5 moles of ethylene oxide, saturated C₁₆ to C₃₀diols, saturated C₁₆ to C₃₀ monoglycerol ethers, saturated C₁₆ to C₃₀ hydroxy fatty acids, and mixtures thereof, having a melting point of at least about 40° C. In one aspect, the moisturizing personal care composition comprises a hydrophobic structuring agent at levels, by weight of the moisturizing personal care composition, of: less than about 20%, less than 10%, and less than 5%. In one aspect, the moisturizing personal care composition comprises a hydrophobic structuring agent at levels by weight of the moisturizing personal care composition of: at least 1%, at least 2%, and at least 3%. Without being limited by theory, it is believed that these structuring agents are useful to assist in the formation of the rheological characteristic of the composition which contributes to the hydrolytic stability of the composition of the present invention. In particular, structuring agents assist in the formation of the liquid crystalline gel-network structures.

The preferred structuring agents of the present invention are selected from the group consisting of stearyl alcohol, cetyl alcohol, behenyl alcohol, stearic acid, palmitic acid, the polyethylene glycol ether of stearyl alcohol having an average of about 1 to about 5 ethylene oxide units, the polyethylene glycol ether of cetyl alcohol having an average of about 1 to about 5 ethylene oxide units, the polyethylene glycol ether of stearyl alcohol having an average of about 2 ethylene oxide units (steareth-2), the polyethylene glycol ether of cetyl alcohol having an average of about 2 ethylene oxide units, steareth-2 and mixtures thereof.

Hydrophilic surfactant is one component of the gel-network. The surfactant, at a minimum, must be hydrophilic enough to disperse in water. Without being limited by theory, it is believed that the hydrophilic surfactant disperses the hydrophobic materials (e.g. the structuring agent), in the water phase. The exact surfactant chosen will depend upon the pH of the composition and the other components present. One example is nonionic surfactants further described in application U.S. Publication No. 2004/0223992. In one aspect, the moisturizing personal care composition comprises a hydrophilic surfactant at levels, by weight of the moisturizing personal care composition, of less than about 10%, less than about 6%, and less than about 3%. In one aspect, the moisturizing personal care composition comprises a hydrophilic surfactant at levels, by weight of the moisturizing personal care composition, of: at least 0.1%, at least 0.2%, and at least 0.3%.

Emulsifiers can be useful in some embodiments of the moisturizing personal care composition. Nonlimiting examples include PROLIPID 141 (glyceryl stearate, behenyl alcohol, palmitic acid, stearic acid, lecithin, lauryl alcohol, myristyl alcohol and cetyl alcohol) and 151 (Glyceryl stearate, cetearyl alcohol, stearic acid, 1-propanamium, 3-amino-N-(2-(hydroxyethyl)-N-N-Dimethyl,N-C(16-18) Acyl Derivatives, Chlorides) from ISP; POLAWAX NF (Emulsifying wax NF), from Croda; and EMULLIUM DELTA (cetyl alcohol, glyceryl stearate, peg-75 stearate, ceteth-20 and steareth-20) from Gattefosse.

The moisturizing personal care composition can comprise one or more benefit agents, as described above or one or more optional ingredients, as described below. Preferably when the moisturizing personal care composition comprises an oil in water emulsion, the benefit agent is dispersed within the skin compatible oil. When the oil in water emulsion comprises a structured oil phase the benefit agent is preferably dispersed within the structured oil phase.

a. Anti-irritation Agent

In one embodiment the moisturizing personal care composition of the present invention further comprises an anti-irritation agent. The anti-irritation agent can be pyrithione or a polyvalent metal salt of pyrithione, or a mixture thereof. Any form of polyvalent metal pyrithione salts may be used, including platelet and needle structures. Preferred salts for use herein include those formed from the polyvalent metals magnesium, barium, bismuth, strontium, copper, zinc, cadmium, zirconium and mixtures thereof, more preferably zinc. Even more preferred for use herein is the zinc salt of 1-hydroxy-2-pyridinethione (known as “zinc pyrithione” or “ZPT”); more preferably ZPT in platelet particle form, wherein the particles have an average size of up to about 20 μm, preferably up to about 5 μm, more preferably up to about 2.5 μm.

The platelet ZPT includes a median particle diameter of about 0.5 microns to about 10, alternatively about 1 to about 5 microns, and alternatively about 3 microns; a mean particle diameter of about 0.5 to about 10 microns, alternatively about 1 to about 5 microns, alternatively about 2 to about 4 microns, and alternatively about 3 microns, and a thickness of about 0.6 to about 15 microns, alternatively about 0.6 to 1 micron, alternatively about 0.6 to about 0.8, and alternatively about 0.6 to about 0.7 microns. The platelet ZPT can also have a span of less than about 5, and alternatively about 1.

Pyridinethione anti-microbial and anti-dandruff agents are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

Preferred embodiments include from 0.01% to 5% of an anti-irritation agent; alternatively from 0.05% to 2%, alternatively from 0.1% to 1%, alternatively from 0.2% to about 0.7%, alternatively about 0.5%.

Those of skill in the art will understand that the anti-irritation agent of the present invention can also have other benefits which may be desirable from a skin care composition, including but not limited to malodor control and/or anti-bacterial benefits depending on whether the composition is left on skin or washed off. Those of skill in the art will understand that the moisturizing composition may be preferred to be at least rinsed off with water after having contacted skin for a set amount of time.

The composition of the present invention optionally includes an effective amount of a zinc salt. Preferred embodiments of the present invention include an effective amount of a zinc salt having an aqueous solubility within the composition of less than about 25%, by weight, at 25° C., more preferably less than about 20%, more preferably less than about 15%. Preferred embodiments of the present invention include from 0.001% to 10% of a zinc salt, more preferably from 0.01% to 5%, more preferably still from 0.1% to 3%. In a preferred embodiment, the zinc salt has an average particle size of from 100 nm to 30 μm.

Examples of zinc salts useful in certain embodiments of the present invention include the following: Zinc aluminate, Zinc carbonate, Zinc oxide and materials containing zinc oxide (i.e., calamine), Zinc phosphates (i.e., orthophosphate and pyrophosphate), Zinc selenide, Zinc sulfide, Zinc silicates (i.e., ortho- and meta-zinc silicates), Zinc silicofluoride, Zinc Borate, Zinc hydroxide and hydroxy sulfate, zinc-containing layered materials and combinations thereof.

In embodiments having an anti-irritation agent and a zinc salt, the ratio of zinc salt to anti-irritation agent is preferably from 5:100 to 5:1; more preferably from about 2:10 to 3:1; more preferably still from 1:2 to 2:1.

Without intending to be bound by theory, it is believed that these anti-irritation agents can provide various benefits including reduction or control of irritation as well as certain malodor control. In one embodiment, the composition further comprises other agents such as malodor control agents. The malodor active of the present invention is capable of providing an antimicrobial benefit. Such malodor actives are capable of destroying microbes, preventing the development of microbes or preventing the pathogenic action of microbes. A safe and effective amount of a malodor active may be added to the composition, at from about 0.001% to about 10%, or from about 0.01% to about 5%, or from about 0.05% to about 2%, or from about 0.1% to about 1%, or from about 0.3% to about 0.7%, or about 0.5% by weight of the composition.

Examples of malodor actives include 13-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chlortetracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline hydrochloride, oxytetracycline hydrochloride, clindamycin hydrochloride, ethambutol hydrochloride, metronidazole hydrochloride, pentamidine hydrochloride, gentamicin sulfate, kanamycin sulfate, lineomycin hydrochloride, methacycline hydrochloride, methenamine hippurate, methenamine mandelate, minocycline hydrochloride, neomycin sulfate, netilmicin sulfate, paromomycin sulfate, streptomycin sulfate, tobramycin sulfate, miconazole hydrochloride, ketaconazole, amanfadine hydrochloride, amanfadine sulfate, octopirox, parachlorometa xylenol, nystatin, tolnaftate, clotrimazole, and mixtures thereof.

According to the method of the invention, a person may shave their face using a product dispensing razor comprising a moisturizing composition with or without the addition of any water (i.e. as a wet shaving device or a dry shaving device) other than the water comprised within the moisturizing composition. This method has the benefit of being environmentally friendly, since shaving may be performed using only the water within the composition itself and since the composition comprises low levels of surfactant. In such a case, since these compositions do not foam, a user may have difficulty tracking the progress of the shaving, that is establishing which area of skin has already been shaved and which has not (hereinafter referred to as “tracking”). As explained above, in such a case, an opacifier or colorant may be added to the moisturizing composition to facilitate tracking. Any water dispersible or water soluble colorant may be used, such as an organic dye, an encapsulated organic dye or a metal oxide pigment.

In another embodiment, a conventional foaming shaving preparation can be applied to the skin prior to shaving with the present device. For the event that a user decides not to shave with a moisturizing composition alone, then a foaming shave preparation may additionally be applied before shaving. The use of two such compositions may allow the provision of benefits which cannot be achieved using a single composition alone.

When mixed with foaming shave preparations which can already be present on the face prior to the use of the present device, the moisturizing compositions used in the method of the invention can give rise to a rich, creamy mixture. It is known that the presence of hydrophobic components, such as oil, may reduce the stability of a foam. Low oil or oil-free moisturizing compositions as defined herein tend not to collapse the subsequently applied foam. This can facilitate better tracking.

Without intending to be bound by theory, it can be preferable to use this device and composition in a wet or dry shave context, without needing to pre-moisturize or hydrate the skin or hairs prior to contact with the razor cartridge or blades. After the shaving step, the user can be instructed to wait for a set amount of time (such as 5 seconds to 120 seconds, or from 20 seconds to 60 seconds, before rinsing or washing off, or, the user can wash / rinse off immediately thereafter. The present composition is designed to provide hydrating benefits and be less irritating to skin, as such, it can be useful in a dry shave or wet shave usage. In one embodiment, the user can be instructed to wait for a set amount of time before rinsing or washing off any remnants of the moisturizing composition from their skin.

According to a further aspect of the invention a kit is provided comprising (a) the product dispensing device of the present invention and (b) a foaming shave preparation. The use of the elements of the kit in the defined way may provide a better shave than the use of a foaming shave preparation alone, because it achieves a different shaving experience.

In one embodiment, the composition contained within the device can be something other than the moisturizing personal care composition disclosed above. In one embodiment, the composition dispensing device contains personal cleansing composition comprising personal cleansing additive, surfactant system of mid-chain branched & linear surfactants (specified structure), and >50% aqueous liquid carrier as disclosed in U.S. Pat. No. 6,335,312, a Personal cleansing composition comprising a cleansing phase (cleansing surfactant+water) and active phase (lipophilic carrier+solid, non-structuring particulates) wherein each phase is physically separated from another by packaging barrier, as disclosed in U.S. Pat. No. 6,673,755 (making the dispensing device a dual chamber device). The composition can also be a striped personal cleansing composition comprising first stripe cleansing phase (surfactant, water, optional other materials) and at least one other stripe comprising benefit phase (>20% hydrophobic material w/Sol. Parameter 5-15, specified rheology) (Sur.+separate hydrophobic benefit phase) as disclosed in US Patent Pub. 2004/ 0057920 or any of the other personal cleaning systems disclosed in the following patent documents: U.S. Pat. No. 7,511,003, US2004/0219119, US2004/0235693, US2004/0248748, US2006/0094628, U52005/0100570, US2005/0192187, US2005/0276768, US2006/0008438, U.S. Pat. No. 7,666,825, U.S. Pat. No. 7,820,609, US2006/021474, U.S. Pat. No. 7,700,528, US2007/0141001, US2007/0137042, US2007/0187274, WO2007/099504, US2008/0242573. Those of skill in the art will understand that these compositions can also include the anti-irritation agent as disclosed above.

2. Composition Dispensing Device

The composition dispensing device of the present invention can be any such device which allows the present composition to be dispense therefrom during the hair removal process. Examples of many types of composition dispensing devices are known. In one embodiment, the composition dispensing device is a composition dispensing razor comprising one or more safety razors.

In one embodiment, the device is an automatic vibrating and/or dispensing razors. For example, U.S. Patent Publ. No. 2008-0289185 to Clark which discloses a razor comprising a fluid delivery system having an electrically actuable dispensing device to control delivery of the fluid, and a control device for controlling actuation of the dispensing device. The control device is in proximity or is touch sensitive and includes a sensor element arranged to be brought into contact with or into close proximity to the skin being shaved during the performance of a shaving stroke. Another suitable device is disclosed in U.S. Pat. No. 7,441,336 to Hawes et al, which discloses an automated razor which has a control device allowing for automated vibration or dispensing when a certain environmental condition is met, such as proximity or touch with the intended surface or electrical conductance. These types of automated dispensing devices can be particularly useful so that the composition can be dispensed at the desired time onto the skin, thereby minimizing wasted product which could otherwise be captured within the device head or elements thereof. Further, using an automated device may be advantageous as users may have a difficult time deciding when to trigger the dispense action during the hair removal process. For example, they may accidently trigger the dispense too early or excessively, causing an undesirably large amount of composition to come out and potentially miss the intended surface for treatment. Automated dispensing, however, is not required, as manual dispensing systems can also be useful for certain purposes.

Non-limiting examples of other composition dispensing devices suitable for use with the present invention include those disclosed in U.S. Patents Publications 2006/00240380 to Chenvainu et al; 2007/0084074 to Szczepanowski et al; U.S. Pat. Nos. 7,127,817; 7,121,754; and 6,789,321. In some of these examples, the product can be dispensed at or about the vicinity of the device head (commonly a razor cartridge).

In one particularly useful embodiment, the composition can be dispensed through an elongated elastomeric contact region comprising a dispensing orifice which allows the composition to spread in a wide strip onto the surface, such as the lumens described in U.S. Patents Publications 2006/00240380 to Chenvainu et al (see inter alia FIG. 17). Similar dispensing systems have also been described in U.S. Ser. No. 61/340299 to Royle et al, filed Mar. 15, 2010. The lumens or dispensing orifice can be particularly useful when dispensing the present moisturizing personal care composition given the specific rheology (thickness and viscosity) desired for use herein. In particular, since the moisturizing personal care composition is desirably thick and viscous, an elongated dispensing orifice or a plurality of orifices oriented to dispense a wide yet thin layer of the composition may be desired. Advantageously, the layer of composition deposited does not excessively run or drip off the surface prior to the treated surface coming in contact with the razor blade or other hair removal head. Further, where the composition is clear or translucent, the user can easily see where they are shaving so they can have fine control to make clean shaven areas such as a beard line. This can be particularly beneficial over devices comprising shaving heads with rotary blades which would not be as capable of allowing for fine control to make clean shaving lines.

In one embodiment, the composition dispensing device comprises a handle connected to a hair removal head, the handle further comprising a cavity for housing said moisturizing personal care composition disposed within the handle, and an actuator adapted to displace moisturizing personal care composition from the cavity to a fluid dispensing member, and wherein said fluid dispensing member comprises an elongated elastomeric contact region forming at least one dispensing orifice which is generally perpendicular to a transverse centerline of the handle. The fluid dispensing member may extend to or adjacent to the bottom portion of the hair removal head allowing for direct contact or near direct contact to a user's skin during application of the hair removal head to skin, such as during a shaving stroke. The fluid dispensing member comprises a fluid dispensing member comprising at least one elongated elastomeric contact region. In one embodiment, the fluid dispensing member also comprises a one-way valve, which can be formed from said elongated elastomeric contact region. Those of skill in the art will understand that the elastomeric material forming the flap valve, slit valve or duckbill valve is such that upon contact with skin, the valve will deform and allow said one or more dispensing orifice(s) to allow fluid to dispense.

In one embodiment, said elongated elastomeric contact region forms a one-way valve which will only allow the composition to exit so entry of undesirable contaminants into the plumbing or cavity of the device is minimized. Non-limiting examples of suitable one-way valves include: check valves such as diaphragm check valves, swing check valves or tilting disc check valves, stop-check valves, lift-check valves, flap valves, slit valves, and/or a duckbill valve. In one embodiment, the fluid dispensing member forms at least one, but optionally two or more dispensing orifices at the dispensing end of the elongated elastomeric contact member for delivering said fluid from the cavity onto skin prior to hair removal. To prevent the fluid from leaking, the fluid flow path, along with any or all of the dispensing orifice(s) may comprise a check valve.

In another embodiment, the cavity can have multiple compartments. For example the cavity can have a first compartment containing said moisturizing personal care composition, and wherein said cavity forms a second compartment for a second composition. In another embodiment, the device comprises multiple cavities, where different compositions can be contained therein. In one embodiment, the second skin care composition is selected from the group consisting of an aftershave, a lotion, a balm, a fragrance, or a mixture thereof. Examples of known dispensing devices which allow for multiple compositions to be contained therein include: U.S. Pat. Nos. 6,986,207; 5,855,066; and 4,129,942. In one embodiment, the moisturizing personal care composition can be used with a device capable of dispensing multiple compositions such as therein described.

a. Hair Removal Head

The hair removal head can include a wide scraping surface such as where the composition dispensing device is used with a depilatory, or a razor cartridge where the device is a shaving razor. The hair removal head may be replaceable or pivotally connected to a cartridge connecting structure. In an aspect, the cartridge connecting structure includes at least one arm to releasably engage the hair removal head.

Where the hair removal head is a razor cartridge the cartridge may also include multiple blades. For example, U.S. Pat. No. 7,168,173 generally describes a Fusion® razor that is commercially available from The Gillette Company which includes a razor cartridge with multiple blades. Additionally, the razor cartridge may include a guard as well as a shaving aid. A variety of razor cartridges can be used in accordance with the present invention. Nonlimiting examples of suitable razor cartridges, with and without fins, guards, and/or shave aids, include those marketed by The Gillette Company under the Fusion®, Venus® product lines as well as those disclosed in U.S. Pat. Nos. 7,197,825, 6,449,849, 6,442,839, 6,301,785, 6,298,558; 6,161,288, and U.S. Patent Publ. 2008/060201.

b. Fluid Dispensing Member

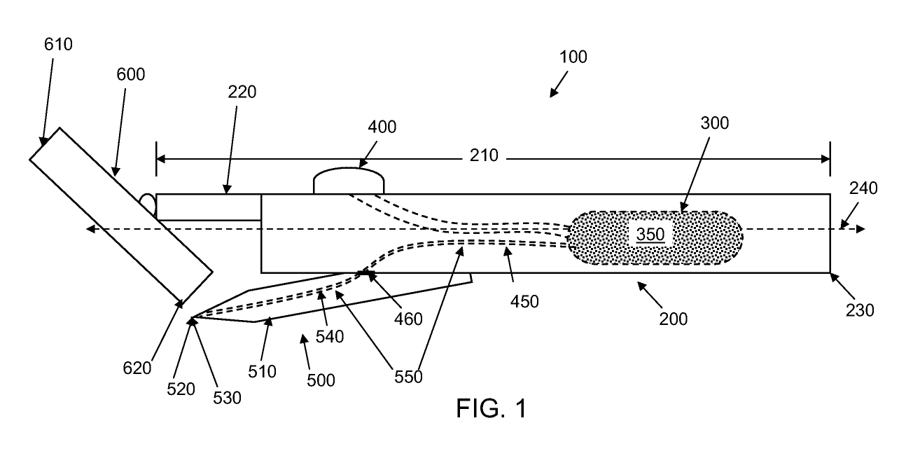

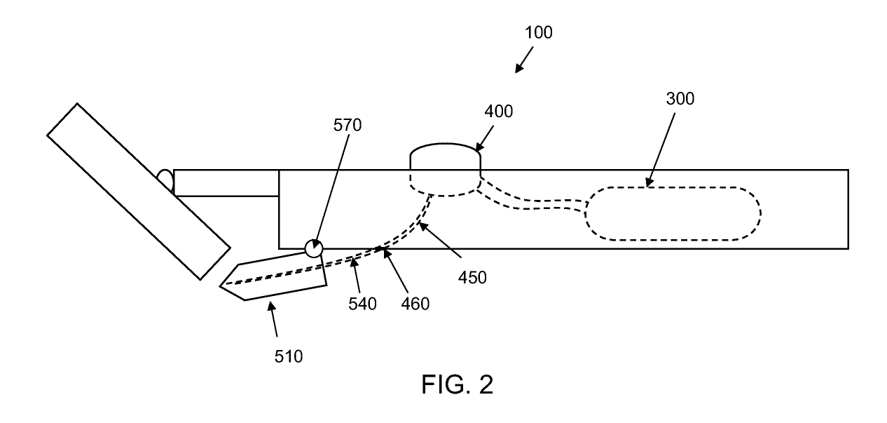

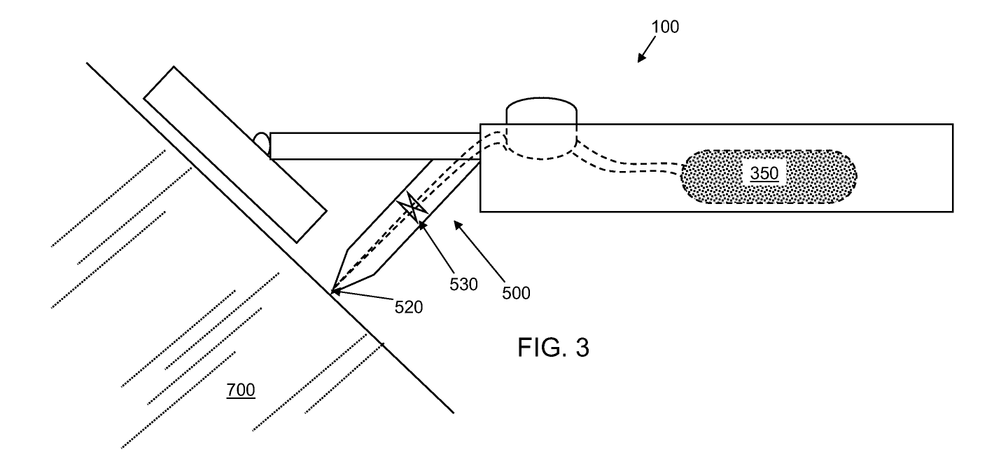

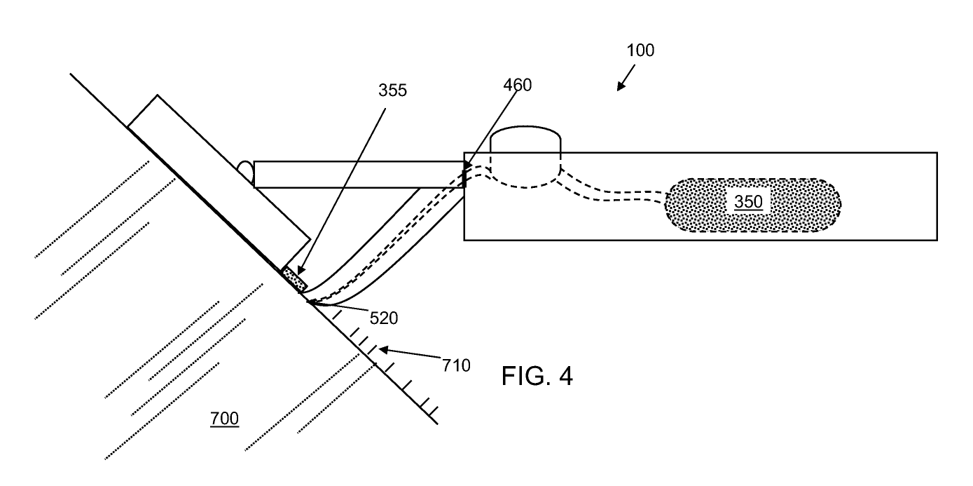

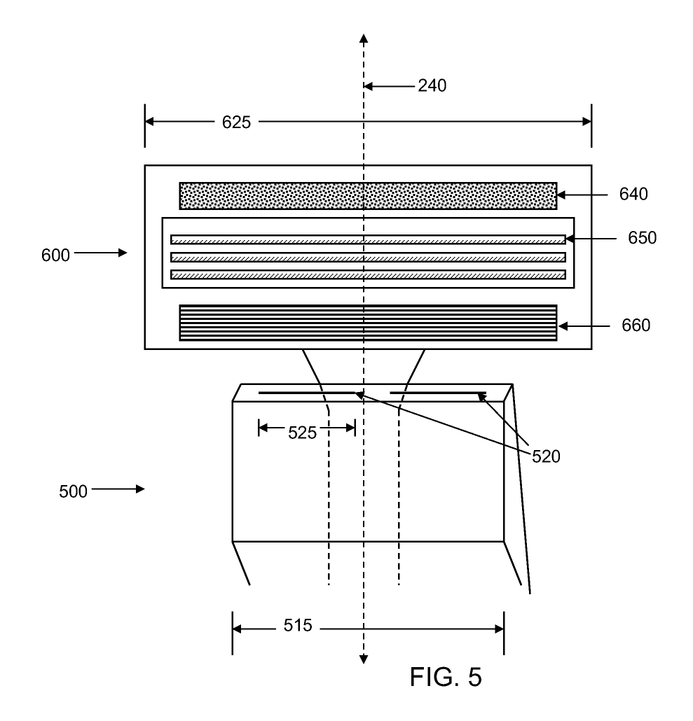

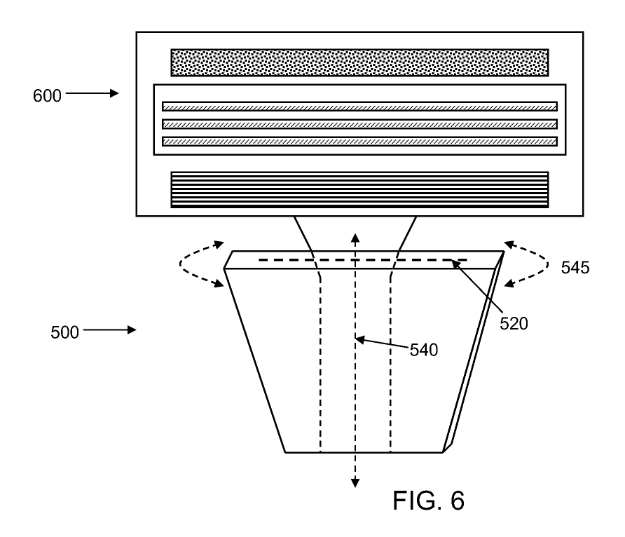

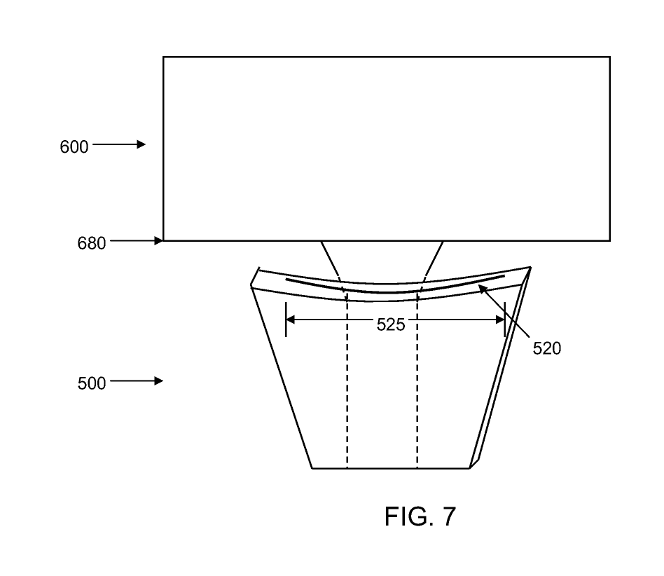

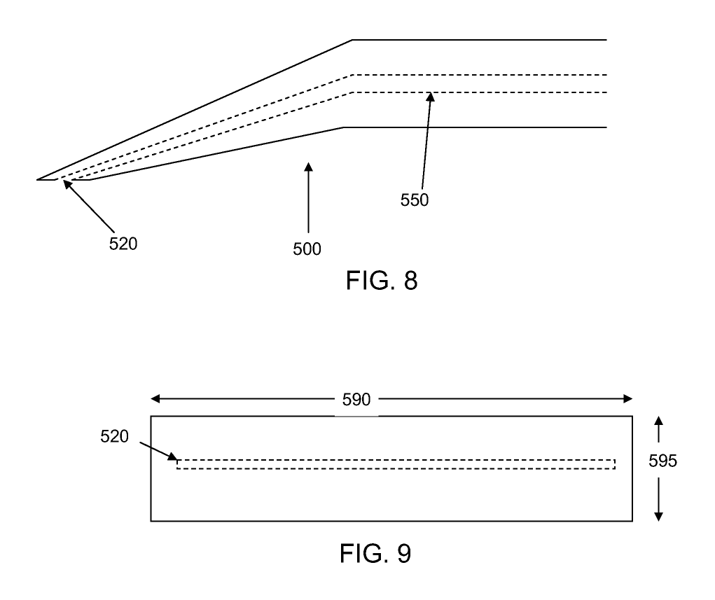

The fluid dispensing member of the present invention comprises an elongated elastomeric contact region. Non-limiting examples of suitable elongated elastomeric contact regions include: dual slit or duckbill valves such as those described in U.S. Patent Pub. 2006/00240380 in FIGS. 1-9 and paragraphs 52 to 58. The present invention, however, does not require dual lumens to be present. Further, the present fluid dispensing member is designed to deliver fluid away from, preferably preceding, the head area of the personal care devices disclosed in the past. By delivering fluid prior to the skin contacting the hair removal head, it allows for broader spreading of the fluid and additional time where the fluid can come into contact with the folds and crevices within the skin. In one embodiment, the fluid dispensing member further comprises a non-elastomeric portion which can precede said elongate elastomeric contact member. The non-elastomeric portion can be formed of the same material as used to form any part of the remainder of the handle. In one embodiment, the one-way valve is not formed in said elongated elastomeric contact member. The one-way valve can be formed in the non-elastomeric portion of said fluid dispensing member or in any portion of said fluid flow path, such as in the supply channel, at the opening, and/or in the fluid dispensing path.

“Elongated” as defined herein means, that the object has a major and a minor axis, wherein the major axis is at least 10 times larger than the minor axis. The elongated portion of the fluid dispensing member has a width (major axis) which is at least 10 times larger than the height. In one embodiment, the width measures from about 2 cm to about 15 cm, alternatively from about 3 cm to about 10 cm, alternatively from about 4 cm to about 8 cm. In another embodiment, the height of the elongated one-way elastomeric valve is about 1 cm, alternatively about 0.5 cm, alternatively from about 0.2 cm, alternatively the elongated one-way elastomeric valve is biased to be in a sealed orientation when not in use. Those of ordinary skill in the art will understand that a check valve may be used in embodiments where the elongated one-way elastomeric valve is not sealed when not in use to minimize product leakage. In another embodiment, the fluid is chosen such that even if the height of the valve is such that it remains unsealed and open when not in use, the fluid is sufficiently viscous and thick that it will not undesirably leak when not being actuated by the user.

“Elastomeric” as defined herein means a material which is generally flexible and deformable. In one embodiment, the elongated elastomeric contact member has a young's modulus of elasticity of from about 0.01 GPa to about 3.5 GPa, alternatively from about 0.02 GPa to about 2 GPa, alternatively from about 0.05 GPa to about 1 GPa, alternatively from about 0.1 GPa to about 0.5 GPa. Non-limiting examples of suitable materials which can be used to form the elastomeric contact member include rubber, silicone, Teflon, and polyethylene. Without intending to be bound by theory, it is believed that by providing an elastomeric material in the fluid dispensing member at the point where the fluid dispensing member would contact skin is particularly useful as it decreases irritation onto skin from a non-elastomeric fluid dispensing member. Further, the elastomeric material allows the tip of the fluid dispensing member to deform to better engage the non symmetric shape of body parts. In one embodiment, the elastomeric material used has a shore hardness of from about 30 to about 40 D units.

“Slit valve” as defined herein means that the valve comprises a closed slit and flow is provided by flexing or deformation of the elastomeric material which causes the slit to open. In general the slit valve is a single piece construction which is free of moving parts. “Duckbilled valve” as defined herein is a type of slit valve, wherein one end of the valve is stretched over the outlet of the fluid dispensing path, conforming itself to the shape of the path, usually round. The other end, the duckbill, retains a natural flattened shape. When a fluid is pumped through the fluid dispensing path, the duckbill' s flattened end opens to permit the pressurized fluid to pass.

When pressure is removed, however, the duckbill end returns to its flattened shape, preventing backflow. Other check valves referred to herein are known in the art.

“Generally perpendicular” as defined herein means that the lateral dimension of the elongated elastomeric contact region forms an angle which is from about 75° to about 90° as measured against the transverse centerline passing through the handle. Since the elongated elastomeric contact region is generally elastic and therefore deformable in nature, this angle is measured when the fluid dispensing region is at rest and not deformed or otherwise manipulated by a user. The elongated elastomeric contact region comprises a contact point where the fluid dispensing member engages the surface (skin). In one embodiment, the contact point forms a straight line. In another embodiment, the contact point forms a concave or convex line. Similarly, “generally parallel” as defined herein means that the two straight lines formed through said objects are parallel or form an angle of from about 0° to about 15° when in a resting position.

The fluid flow path terminates at least one fluid dispensing orifice. In one embodiment, more than one fluid dispensing orifice is provided. The fluid dispensing orifice is formed of the elastomeric material used to form the elongated elastomeric contact member. Preferably, the fluid dispensing orifice has a wide and narrow shape similar to the fluid dispensing member but the fluid dispensing orifice can also be of different shapes. In one embodiment, the fluid dispensing member comprises a plurality of fluid dispensing orifices which are spaced out along the width of the elongated elastomeric contact member such that when fluid is dispensed, a wide flat application of fluid can still be deposited. The fluid dispensing orifices can be round, oval, triangular, square, rectangular in shape, or combinations thereof. In one embodiment, the portion of the elongated elastomeric contact member leading up to the fluid dispensing orifice forms a tapered exit channel, the tapering can be tapered in, or tapered out. Without intending to be bound by theory, it is believed that a tapered exit channel, particularly one that tapers out so the cross sectional area of the orifice is larger than the cross sectional area of the channel leading to the orifice, can be useful to ease in removal of any fluid which can reside in the vicinity of the orifice after use. A tapered in exit channel may be useful to minimize exposure of the fluid to contact with air, thereby minimizing fluid dry out.

c. Actuator

As explained above, the actuator can be manual or automatic pump (battery powered or via an external power source). The pump includes a wall, either movable or rigid, upon which force is acted upon to move the fluid through. In the case of a movable wall, the movable wall may be located on one or more of an upper or lower surface of the handle. For a rigid wall, the force causes the movement of non-rigid sidewalls of the pump to move a fluid through to the channel.

In one embodiment, the actuator is a manually-actuated pump which can reside on the handle. In another embodiment, the actuator is automated and can be powered by a battery or external power source. In yet further embodiments, the actuator comprises a pump which is actuated by movement of the shaving head (such as where depression of the head or rotation of the head about the pivot axis), actuates the pump. In yet another embodiment, the fluid dispensing member itself can be spring loaded and retractable upon contact with a surface such as skin such that the movement of the fluid dispensing member can act as the actuation to actuate the pump. Those of skill in the art will understand that in this type of embodiment, it could be preferred to have the elongated elastomeric contact region extend beyond the general plane of the shaving head such that when the device is brought into contact with a surface (such as skin) the fluid dispensing member will be pushed back towards the razor prior to surface contact with the shaving head. The movement of the fluid dispensing member can then actuate the pump permitting fluid to escape or be driven out of the cavity through the fluid flow path, out of the at least one dispensing orifice, ultimately onto the skin.

Other features and advantages of the invention will be apparent from the description and drawings, and from the claims.

3. Figures

FIGS. 1-4 provide side views of composition dispensing devices which are in accordance with at least one embodiment of the present invention. Composition dispensing device (100) is suitable for dispensing a fluid during the hair removal process, such as shaving. The composition dispensing device (100) includes a handle (200) and a hair removal head (600), such as a disposable razor cartridge. Hair removal head (600) includes a top portion (610) and a bottom portion (620). Those of skill in the art will understand that the composition dispensing device can be a wet or dry, manual or powered razor, having straight or rotary blades. In addition, the composition dispensing device can be used with a depilatory, therefore not requiring the use of razor. The handle (200) has a length (210) that extends from a proximal end (220) to a distal end (230) and a transverse centerline (240) which runs along the central axis of the handle. The handle comprises a cavity (300) for housing a fluid (350) disposed within the handle, and an actuator (400) adapted to displace the fluid from the cavity through a supply channel (450) to an opening (460) formed in said handle, such as towards the proximal end of the handle.

The composition dispensing device (100) includes a fluid dispensing member (500) comprising an elongated elastomeric contact region (510) forming at least one dispensing orifice (520) in fluid communication with said opening (460) formed in said handle (200). Said elongated elastomeric contact region comprises a lateral dimension (515), shown in FIG. 5, which is generally perpendicular to said transverse centerline (240) of the handle. The hair removal head also has a lateral dimension (625) which his generally perpendicular to said transverse centerline. In one embodiment, the ratio of the lateral dimension of the elongated elastomeric contact region (515) to the lateral dimension of the hair removal head (625) is from 1:10 to about 1.5:1, alternatively from about 0.5:1 to about 1:1. Without intending to be bound by theory, it is believed that by providing an elongated elastomeric contact region which is laterally sized with respect to the hair removal head as recited herein, the fluid dispensed from said at fluid dispensing member covers a sufficiently broad portion of said hair removal head to provide suitable product spreading over skin and into cracks and corners of the skin. The fluid dispensing member is in fluid communication with said cavity via said opening, forming a fluid dispensing path (540), wherein said supply channel and said fluid dispensing path form a fluid flow path (550).

In one embodiment, the device includes at least one one-way valve (530) located at some point along said fluid flow path. As explained above, in some embodiments, said elongated elastomeric contact point forms said one-way valve. Additional one-way valves can also be included along the fluid flow path as desired.

The actuators shown in FIGS. 1-4 are manual pumps but automatic pumps can also be included. The actuators shown in FIGS. 1-4, which can be manual or automatic, and may include pumps which can be stacked (and substantially flat) components and particularly a movable wall that acts to activate the flow of fluid from the cavity through channel and to the opening. A pump suitable for use in the present invention is disclosed in U.S. Pat. No. 5,993,180. In particular, this pump includes a pump chamber bounded by the movable wall, an inlet channel and an outlet channel, both of which are connected to the pump chamber, an inlet valve for closing the inlet channel, and an outlet valve for closing the outlet channel. In most instances, the pump may be actuated by the pressure exerted by a user's finger such that the user may easily determine the requisite amount of fluid for one or more shaving strokes. Because the valves of the pump are automatically opened when pressure is applied by the user's finger pressure, the fluid can be dispensed in controlled and metered quantities without relying on judgment or dexterity of the user. It is also possible to place one or more movable walls of the pump on an upper surface or lower surface of the razor depending on a user's preference. The actuator of FIG. 1 is shown providing a feed into the cavity. This feed can be application of pressure or another impulse which will drive fluid through said fluid flow path out to the fluid dispensing member. The actuator shown in FIG. 2, however, has a receiving chamber where fluid is transferred prior to entering the supply channel and passing into the fluid dispensing member. These and other actuators and pumps which are known in the art for use in personal care devices which dispense fluids can be used in accordance with the present invention.

The cavity (300), or at least a container/sachet within the cavity (300), contains the fluid (350) to be dispensed during the hair removal process. In one embodiment, the fluid (350) in the cavity or container is refillable or replaceable.

FIG. 2 provides a side view, where the fluid dispensing member is pivotably attached to said handle via a hinge member (570) positioned on said handle. Further, as shown in this figure, in one embodiment, a portion of the fluid flow path, such as the fluid dispensing path (540) can be exposed upon exiting said opening (460) formed in said handle.

FIGS. 3 and 4 provide two exemplary side views of a composition dispensing device wherein the fluid dispensing member is shown deforming. In FIG. 3, the fluid dispensing member (500) is shown extending beyond the general facial plan formed by said hair removal head. Since the fluid dispensing member comprises an elastomeric contact region, the portion of the fluid dispensing member which extends beyond the plane of the hair removal head would deform, as generally depicted in FIG. 4 when the device comes in contact with a surface, such as skin (700). In FIG. 4, a fluid dispensing member is deformed when the device is in an “in-use” position, allowing fluid to exit the at least one dispensing orifice (520) formed in the elongated elastomeric contact region. The elongated elastomeric contact region can flex toward the hair removal head, flex away from the hair removal head and can even come into contact with a portion of the hair removal head, all depending on the movement of the device with respect to the surface. A volume of fluid (355) is deposited onto the skin and the hair removal head is moved in a downward trajectory along the skin to remove hairs (710) which have been treated with said fluid. Further, the one-way valve shown in FIG. 3 is positioned along the fluid flow path but not at the point where the fluid dispensing member forms said at least one dispensing orifice.

The cartridge (600) attaches to the rear surface of a housing (not shown) by a cartridge connecting structure (not shown). The cartridge connecting structure may include one or more arms that extend to provide pivotal support of the housing. Alternatively, the cartridge connecting structure may include an ejection mechanism (e.g., a button) to disengage the housing from the cartridge connecting structure. Non-limiting examples of suitable housings and cartridge connecting structures are described in: U.S. Pat. Nos. 7,197,825, 5,822,869, 6,161,287, and 5,784,790.

The razor cartridge (300) may also include a guard (330) or lubricating strip located between the top portion (204) and bottom portion (206). The guard (330) is useful for stretching the skin's surface immediately prior to engagement with the blade or a first blade (when more than one blade is present). This guard (330) may typically comprise an elastomeric member to allow for an engagement that is comfortable to a user. U.S. Pat. No. 7,168,173 discloses a suitable razor cartridge and elastomeric material without the apertures. The elastomeric material can be selected as desired. Typically, the elastomeric material used is a block copolymer (or other suitable materials), e.g., having a durometer between 28 and 60 Shore A.

The shaving aid, also known as a lubricating strip, on the other hand, provides an additional treatment to the skin after contact between the fluid and the skin has occurred. The lubricating strip may contain the same or additional skin ingredients to those that are present in the fluid. Suitable shave aids/lubricating strips are disclosed in U.S. Pat. Nos. 7,069,658, 6,944,952, 6,594,904, 6,182,365, D424,745, 6,185,822, 6,298,558 and 5,113,585. The cartridge connecting structure (312) may be releasably engaged from the handle (300), as disclosed in U.S. Pat. Nos. D533,684, 5,918,369, and 7,168,173. This disengagement of these two components allows for replacement of razor cartridges as the continued use of such cartridges causes blade dulling. Thus, such cartridges are replaceable and disposable at will by the user.

FIG. 5 provides a bottom planar view of a portion of a composition dispensing device in accordance with at least one embodiment of the present invention. Hair removal head (600) is shown as a razor cartridge with a plurality of blades (650) and a shaving aid (640) as well as a guard (660). The razor cartridge is shown having a lateral dimension which can measure any length typically used for conventional straight blade wet razor cartridges, for example from about 2 cm to about 10 cm, alternatively from about 3 cm to about 8 cm, alternatively from about 4 cm to about 7 cm. A transverse centerline of the handle is shown as (240). Said elongated elastomeric contact region (500) comprises a lateral dimension (515) which is generally perpendicular to said transverse centerline. In this embodiment, two fluid dispensing orifices are shown (520). Those of skill in the art will understand that different fluid dispensing orifice configurations are within the scope of the invention. FIG. 5 shows two fluid dispensing orifices which are equal in length and are positioned linear to one another. The lengths can vary and the orifices can be staggered so they do not sit on the same line. Further, although the at least one fluid dispensing orifice is shown being generally parallel to the angle of the razor cartridge and/or blades, the orifice can be angled. The lateral dimension of the at least one fluid dispensing orifice (525) is measured as the greatest lateral distance covered by the orifice, regardless of the angle upon which the orifice sits with respect to the razor cartridge and/or blades. In another embodiment, the at least one fluid dispensing orifice can have a curved or wavy line shape. In one embodiment, the ratio of the lateral dimension of the at least one fluid dispensing orifice (525) to the lateral dimension of the hair removal head (625) is from about 1:10 to about 1:1, alternatively from about 1:5 to about 1:2.

FIG. 6 provides a bottom planar view of a portion of a composition dispensing device in accordance with at least one embodiment of the present invention. The elongated elastomeric contact member is shown with a transverse central axis (540). The elongated elastomeric contact member, being deformable and elastic in nature can twist, bend, compress and stretch as needed. In this embodiment, the elongated elastomeric contact member has a rotation path (545) showing the ability of the elongated elastomeric contact member to rotate about said transverse central axis. In this embodiment, the portion of the elongated elastomeric contact member which forms the at least one fluid dispensing orifice (520) shown in a sealed position, has a greater lateral dimension than the portion of the elongated elastomeric contact member which would be closer to the handle. Those of skill in the art will understand that the elongated elastomeric contact member can have a constant, increasing or decreasing lateral dimension as the lateral dimension is measured from the distal end to the proximal end (towards the handle).

FIG. 7 shows another composition dispensing device in accordance with at least one embodiment of the present invention. In this embodiment, both the tip of the fluid dispensing member (500) and the at least one fluid dispensing orifice (520) are concave shaped so they can contour to body parts easier. The at least one fluid dispensing orifice is shown having a lateral dimension (525). This could be particularly preferable for female composition dispensing devices which are designed for use on the leg or arms. In this embodiment, the hair removal head (600) is shown having a scraping edge (680). The hair removal head can also be a razor cartridge as described above.

In one embodiment, the hair removal head has a skin contacting edge which is flat, concave or convex. Those of skill in the art will understand that different shapes for the skin contacting edge can be preferred based on the desired part of the body upon which the device is intended for use. For example, a composition dispensing device intended for use on the face may have an applicator having a straight edge. A composition dispensing device intended for use on legs may have an applicator having a concave edge. Non-limiting examples of suitable head configurations are disclosed in U.S. Pat. Nos. D399,601, D203,892, and 651,420 to Haglock; U.S. Pat. Nos. 3,088,470, 3,858,985, and 2004 0168743A1 to Garwood; WO Publ. No. 97/18043A1 to Weiss; and GB 1 390 153 to Laboratorio Guidotti & C. S.p.A.

FIG. 8 provides an exploded side view of a fluid dispensing member (500) wherein the fluid dispensing member has an angled and tapered distal region (extending away from the handle). An fluid dispensing orifice (520) is shown in fluid communication with the fluid flow path (550). In one embodiment, a check valve is provided along the fluid flow path. In another embodiment, the fluid dispensing orifice can include a flap or be designed to close when not in use. The fluid dispensing orifice could then act as a one-way valve as described above. In one embodiment the fluid flow path has a constant cross sectional area or a varying cross sectional area. The fluid flow path shown in FIG. 8 is tapered as it approaches the fluid dispensing orifice.

FIG. 9 provides a planar view of the contact region which engages skin during use of a fluid dispensing member. The fluid dispensing orifice (520) is shown in dashed lines as it can be sealed when not in use. The width of the fluid dispensing member (590) is shown as well as the height of the fluid dispensing member (595). In one embodiment, the fluid dispensing orifice has a width of from about 2 cm to about 15 cm, alternatively from about 3 cm to about 10 cm, alternatively from about 4 cm to about 8 cm. Where numerous fluid dispensing members are provided, the width can be even smaller, as low as about 0.2 cm, or about 0.5 cm, or about 1 cm. The width of the fluid dispensing orifice is preferably 0 cm when the device is in a sealed state (not in use) but the width can change when the orifice is opened and can be from about 0.02 cm to about 0.5 cm, alternatively from about 0.05 cm to about 0.3 cm, alternatively from about 0.1 cm to about 0.2 cm. In one embodiment, the fluid dispensing orifice is not 0 cm when not in use. In this embodiment, a check valve can be included somewhere along the fluid flow path to control movement of the fluid before it reaches the fluid dispensing orifice. In another embodiment, the fluid dispensing orifice comprising a width of from about 0.5 mm to about 10 mm, or from about 1 mm to about 3 mm, and a length of from about 20 mm to about 80 mm, or from about 30 mm to about 70 mm, alternatively from about 40 mm to about 50 mm.

4. Methods of Use

As explained above, the present device is designed for use in the hair removal process, such as when shaving. One embodiment of the present invention provides for a method of removing hair from skin comprising the steps of: providing a composition dispensing device containing the moisturizing personal care composition described herein; actuating said composition dispensing device to dispense said composition; contacting said composition onto a portion of skin to be treated to form a prepared surface; and contacting said prepared surface with the composition dispensing device to form a treated surface.

Another embodiment, further comprises a step of wetting said portion of skin to be treated either before contacting said composition onto a portion of skin or after contacting said prepared surface with the composition dispensing device to form a treated surface. The process can also include a step of leaving the treated surface as is, without further washing or rinsing, after the hair removal step.

In another embodiment, the composition is dispenses from the device directly onto skin from the dispensing member of said composition dispensing device. This step can be by manually triggering an actuator, or by an automated control device which senses when the device is in proximity or in contact with the surface to be treated. The composition could also be dispensed onto a portion of the device which is then contacted to the skin to apply the composition but this is not necessary where dispensing directly on to skin is possible.

In yet another embodiment, the steps of contacting said composition onto the skin and contacting said treated surface with the razor blade can occur simultaneously.

In one embodiment, the device is used in a dry shave context where water or other shave preparations are not used to pre-wet the skin. Water can still be used, however, after the dry shave to wash of any shave debris and remaining moisturizing composition. Yet another embodiment provides for a further step of applying a second skin care composition onto the treated surface, such as a post-shave composition. These and other methods of use of the present device in a grooming context are within the scope of the present invention. 5. Moisturizing Personal Care Composition Example

| Ingredient | | | | | | | |
|---|---|---|---|---|---|---|---|
| (by weight %) | 1 | 2 | 3 | 4 | 5 | 6 | 7 |
| Hydroxypropyl Starch | 3.5 | 4.0 | 3.5 | 3.5 | 4.0 | 4.0 | 3.5 |
| Phosphate^{1.} | | | | | | | |
| Emulsifying Wax NF^{2.} | 2.75 | 3.0 | 2.75 | — | 3.0 | 3.0 | 1 |
| Tween 60^{3.} | — | — | — | 0.5 | — | — | — |
| Cetyl Alcohol | — | — | — | 0.4 | — | — | — |
| Stearyl Alcohol | — | — | — | 0.4 | — | — | — |
| Fragrance | 1.0 | 1.0 | 1.0 | 1.0 | 1.0 | 1.0 | 1.0 |
| Preservatives | 0.8 | 0.8 | 0.8 | 0.8 | 0.8 | 0.8 | 0.8 |
| Water | Q.S. | Q.S. | Q.S. | Q.S. | Q.S. | Q.S. | Q.S. |
| Petrolatum^{4.} | 15 | 25 | 20 | 20 | 15 | | 35 |
| Mineral Oil^{5.} | — | — | 5 | — | — | — | — |
| Jojoba Oil^{6.} | — | 5 | — | — | — | — | — |
| Silicone Fluid^{7.} | — | — | — | — | 2 | — | — |
| Gelled Mineral Oil^{8.} | — | — | — | — | — | 15 | — |
| ZPT | 0.5 | — | 1.0 | — | 0.5 | — | 3 |

^{1.}Structure XL available from National Starch;
^{2.}Polawax available from Croda;
^{3.}Polysorbate-60 available from ISP;
^{4.}Superwhite Protopet available from WITC;
^{5.}Hydrobrite 1000 PO White MO available from WITCO;
^{6.}Lipovol J available from Lipo;
^{7.}50 cstk available from Dow Corning;
^{8.}Versagel M750 available from Penreco.

The in-shower body lotion composition of Example 1-7 can be prepared by conventional formulation and mixing techniques. One such example is shown below, although a variety of orders of addition can be used to formulate useable products. Prepare the aqueous phase composition by first dispersing the hydroxypropyl starch phosphate in water. Add gel network phase (emulsifying wax or tween 80/cetyl alcohol/stearyl alcohol blend) and heat to 160° F. Place mixing vessel in a water bath to cool to under 100° F. Add fragrance. Premix all lipids at 160° F. Add to the aqueous phase (<80° F.) with increased agitation. (In the case of examples with multiple lipids, the lipids can be premixed or not, depending upon the desired outcome.) Add preservatives and agitate until product is smooth.

TABLE 7
In-shower body lotion composition of the Present Invention

| | Example# | | | |
|---|---|---|---|---|
| Ingredient (by weight %) | 8 | 9 | 10 | 11 |
| Sepigel 305^{1.} | — | 0.5 | — | — |
| Hydroxypropyl Starch Phosphate^{2.} | 3.5 | 2.5 | 3.5 | 3.5 |
| Emulsifying Wax NF^{3.} | 3.0 | 2.25 | 3.0 | 2.0- |
| Fragrance | 1.0 | 1.0 | 1.0 | 1.20 |
| Preservatives | 0.8 | 0.8 | 0.8 | 0.65 |
| Water | Q.S. | Q.S. | Q.S. | Q.S. |
| Petrolatum^{4.} | 20 | — | — | 25 |
| Mineral Oil^{5.} | — | — | 5 | — |
| G-2180 Petrolatum^{6.} | — | 25 | 20 | — |
| Dimethicone | — | — | — | 2 |
| Shea Butter | — | — | — | .01 |
| Interference Pigments^{7.} | — | — | — | 0.7 |

^{1.}available from Seppic;
^{2.}Structure XL available from National Starch;
^{3.}Polawax from Croda;
^{4.}Superwhite Protopet available from WITCO;
^{5.}Hydrobrite 1000 PO White MO available from WITCO;
^{6.}available from Crompton;
^{7.}KTZ Interval Gold 11s10 available from Kobo Products, Inc.

The in-shower body lotion composition of Example 8-11 can be prepared by conventional formulation and mixing techniques. One such example is shown below, although a variety of orders of addition can be used to formulate useable products. First, prepare the aqueous phase composition by dispersing the hydroxypropyl starch phosphate in water. Add gel network phase (emulsifying wax or tween 80/cetyl alcohol/stearyl alcohol blend) and heat to 160° F. Place mixing vessel in a water bath to cool to under 100° F. Add fragrance. Add the lipid(s) (preheated to 160° F.) to the aqueous phase (<80° F.) with increased agitation. (In the case of examples with multiple lipids, the lipids can be premixed or not, depending upon the desired outcome.) Add preservatives and agitate until product is smooth.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern. Except as otherwise noted, the articles “a,” “an,” and “the” mean “one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition dispensing device comprising: a first chamber and a moisturizing personal care composition contained within said first chamber, said moisturizing personal care composition comprising: an oil-containing composition comprising: a) an aqueous phase; b) from about 1 to about 80 weight percent of a structured oil phase; c) a gel-network phase comprising a non-ionic hydrophilic surfactant and a hydrophobic structuring agent; and d) an aqueous phase stability agent wherein said structured oil phase has a shear index of less than 0.8.

2. The composition dispensing device of claim 1, wherein the moisturizing personal care composition further comprises at least 10 weight percent of said structured oil phase.

3. The composition dispensing device of claim 1, wherein the moisturizing personal care composition further comprises at least 20 weight percent of said structured oil phase.

4. The composition dispensing device of claim 1, wherein said structured oil phase is petrolatum.

5. The composition dispensing device of claim 1, wherein said gel-network phase is present in an amount from about 0.5 to about 20 weight percent of said composition.

6. The composition dispensing device of claim 1, wherein said aqueous phase stability agent is a modified starch.

7. The composition dispensing device of claim 1, wherein said moisturizing personal care composition has a turbidity value of below about 320 NTU.

8. The composition dispensing device of claim 1, wherein the composition dispensing device comprises a handle connected to a hair removal head, the handle further comprising a cavity for housing said moisturizing personal care composition disposed within the handle, and an actuator adapted to displace moisturizing personal care composition from the cavity to a fluid dispensing member, and wherein said fluid dispensing member comprises an elongated elastomeric contact region forming at least one dispensing orifice which is generally perpendicular to a transverse centerline of the handle.

9. The composition dispensing device of claim 8, wherein said elongated elastomeric contact region forms a one-way valve.

10. The composition dispensing device of claim 9, wherein said one-way valve is selected from a flap valve, a slit valve, a duckbill valve, and a combination thereof.

11. The composition dispensing device of claim 10, wherein said cavity forms a first compartment containing said moisturizing personal care composition, and wherein said cavity forms a second compartment for a second composition.

12. The composition dispensing device of claim 11, wherein said second skin care composition is selected from the group consisting of an aftershave, a lotion, a balm, a fragrance, or a mixture thereof.

13. A method of removing hair from skin comprising the steps of:
a. providing a composition dispensing device containing a moisturizing personal care composition as recited in claim 1.
b. actuating said composition dispensing device to dispense said composition;
c. contacting said composition onto a portion of skin to be treated to form a prepared surface; and
d. contacting said prepared surface with composition dispensing device to form a treated surface.

14. The method of claim 13, further comprising a step of wetting said portion of skin to be treated either before step c) or after step d).

15. The method of claim 14, wherein said step b) dispenses said composition directly onto skin.

16. The method of claim 14, wherein said steps c) and d) occur simultaneously.

17. The method of claim 14, wherein the portion of skin to be treated is not pre-wetted with water or a shave preparation before step b.

18. The composition dispensing device of claim 1, wherein said composition further comprises from about 0.01% to 5% of an anti-irritation agent selected from a pyrithione, a polyvalent metal salt of pyrithione, and a mixture thereof.

19. The composition dispensing device of claim 19, wherein said anti-irritation agent comprises zinc pyrithione, wherein said zinc pyrithione is in the form of a platelet having a median particle diameter of about 0.5 microns to about 10, a mean particle diameter of about 0.5 to about 10 microns, and a thickness of about 0.6 to about 15 microns.

20. The composition dispensing device of claim 19, further comprising from 0.001% to 10% of a zinc salt.
